(19) United States
(12) Patent Application Publication
(10) Pub. No.: US 2009/0259296 A1
(43) Pub. Date: Oct. 15, 2009

(54) Gate Cannulation Apparatus and Methods

(75) Inventors: Gregory McIff, Santa Rosa, CA (US); Dwayne S. Yamasaki, St. Augustine, FL (US); David Simon, Boulder, CO (US); Andrew Bzostek, Louisville, CO (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/100,587

(22) Filed: Apr. 10, 2008

(51) Int. Cl. A61F 2/06

(52) U.S. Cl. 623/1.34; 623/1.35; 128/898

(57) ABSTRACT

A representation of a prosthesis opening is created and a device having a marker is tracked through the opening while monitoring the relative positions of the opening representation and the marker on a display. In one alternative, a representation is made on a display of the center of the contralateral stump opening and an electromagnetic marker coil that is secured to an endovascular delivery device, and the marker and delivery device are guided into the opening, while monitoring the relative positions of the opening center and the electromagnetic marker coil representations. In another alternative, one or more markers are positioned in the vicinity of the prosthesis opening and a device having a marker is tracked to the one or more markers and through the opening where the device is one of a guidewire and a catheter, while monitoring the relative position of the markers on a display.

### FIELD OF THE INVENTION

The invention relates to endovasularly delivered prosthesis and more particularly to cannulating the gate of a prosthesis such as a bifurcated stent-graft.

### BACKGROUND OF THE INVENTION

Tubular prostheses such as stents, grafts, and stent-grafts (e.g., stents having an inner and/or outer covering comprising graft material and which may be referred to as covered stents) have been widely used in treating abnormalities in passageways in the human body. In vascular applications, these devices often are used to replace or bypass occluded, diseased or damaged blood vessels such as stenotic or aneurysmal vessels. For example, it is well known to use stent-grafts, which comprise biocompatible graft material (e.g., Dacron® or expanded polytetrafluoroethylene (ePTFE)) supported by a framework (e.g., one or more stent or stent-like structures), to treat or isolate aneurysms. The framework provides mechanical support and the graft material or liner provides a blood barrier.

Aneurysms generally involve abnormal widening of a duct or canal such as a blood vessel and generally appear in the form of a sac formed by the abnormal dilation of the duct or vessel wall. The abnormally dilated wall typically is weakened and susceptible to rupture. Aneurysms can occur in blood vessels such as in the abdominal aorta where the aneurysm generally extends below the renal arteries distally to or toward the iliac arteries.

In treating an aneurysm with a stent-graft, the stent-graft typically is placed so that one end of the stent-graft is situated proximally or upstream of the diseased portion of the vessel and the other end of the stent-graft is situated distally or downstream of the diseased portion of the vessel. In this manner, the stent-graft spans across and extends through the aneurysmal sac and beyond the proximal and distal ends thereof to replace or bypass the weakened portion. The graft material typically forms a blood impervious lumen to facilitate endovascular exclusion of the aneurysm.

Such prostheses can be implanted in an open surgical procedure or with a minimally invasive endovascular approach. When the prosthesis is a stent-graft, a minimally invasive endovascular approach is preferred by many physicians over traditional open surgery techniques where the diseased vessel is surgically opened, and a graft is sutured into position such that it bypasses an aneurysm. The endovascular approach, which has been used to deliver stents, grafts, and stent grafts, generally involves cutting through the skin to access a lumen of the vasculature. Alternatively, lumenar or vascular access may be achieved percutaneously via successive dilation at a less traumatic entry point. Once access is achieved, the prosthesis (e.g., a stent-graft) can be routed through the vasculature to the target site. For example, a stent-graft delivery catheter loaded with a stent-graft can be percutaneously introduced into the vasculature (e.g., into a femoral artery) and the stent-graft delivered endovascularly across the aneurysm where it is deployed.

When using a balloon expandable stent-graft, balloon catheters generally are used to expand the stent-graft after it is positioned at the target site. When, however, a self-expanding stent-graft is used, the stent-graft generally is radially compressed or folded and placed at the distal end of a sheath or delivery catheter. Upon retraction or removal of the sheath or catheter at the target site, the stent-graft self-expands.

More specifically, a delivery catheter having coaxial inner and outer tubes arranged for relative axial movement therebetween can be used and loaded with a compressed self-expanding stent-graft. The stent-graft is positioned within the distal end of the outer tube (sheath) and in front of a stop fixed to distal end of the inner tube. Once the catheter is positioned for deployment of the stent-graft at the target site, the inner tube is held stationary and the outer tube (sheath) withdrawn so that the stent-graft is gradually exposed and allowed to expand. The inner tube or plunger prevents the stent-graft from moving back as the outer tube or sheath is withdrawn. An exemplary stent-graft delivery system is described in U.S. Patent Application Publication No. 2004/0093063, which published on May 13, 2004 to Wright et al. and is entitled Controlled Deployment Delivery System, the disclosure of which is hereby incorporated herein in its entirety by reference.

Regarding proximal and distal positions referenced herein, the proximal end of a prosthesis (e.g., stent-graft) is the end closest to the heart (by way of blood flow) whereas the distal end is the end furthest away from the heart during deployment. In contrast, the distal end of a catheter is usually identified as the end that is farthest from the operator, while the proximal end of the catheter is the end nearest the operator.

Although the endovascular approach is much less invasive, and usually requires less recovery time and involves less risk of complication as compared to open surgery, among the challenges with the approach is positioning the prosthesis and/or locating the prosthesis position.

Generally speaking, physicians often use fluoroscopic imaging techniques to confirm prosthesis position before and during deployment. This approach requires one to administer a radiopaque substance, which generally is referred to as a contrast medium, agent or dye, into the patient so that it reaches the area to be visualized (e.g., the renal arteries). A catheter can be introduced through the femoral artery in the groin of the patient and endovascularly advanced to the vicinity of the renals. The fluoroscopic images of the transient contrast agent in the blood, which can be still images or real-time motion images, allow two dimensional visualization of the location of the renals. The use of X-rays, however, requires that the potential risks from a procedure be carefully balanced with the benefits of the procedure to the patient. While physicians always try to use low dose rates during fluoroscopy, the duration of a procedure may be such that it results in a relatively high absorbed dose to the patient and physician. Patients who cannot tolerate contrast enhanced imaging or physicians who must or wish to reduce radiation exposure need an alternative approach.

Among the challenges in bifurcated stent-graft delivery to an abdominal aortic aneurysm is cannulating the contralateral gate of the stent-graft after the main body section of the stent-graft is deployed. Specifically, inserting a 0.035 mm guidewire, which serves to guide a contralateral leg catheter, into the stent-graft's contralateral gate, which typically has a 1 cm diameter, when the contralateral gate is disposed in an abdominal aortic aneurysm, which typically has a diameter of about 4.5-8 cm and in some cases can be as large as 9-10 cm, can be difficult. Even with the assistance of two-dimensional fluoroscopy, the image may lead an operator to believe that the guidewire has passed into the gate, when in fact it is positioned behind, in front of, or along the side of the stent-graft's contralateral short leg or stump.

Accordingly, there remains a need to develop and/or improve prosthesis positioning and locating apparatus and methods for endolumenal or endovascular applications.

### SUMMARY OF THE INVENTION

The present invention involves improvements in gate cannulation methods and apparatus.

In one embodiment according to the invention, a method of cannulating the contralateral stump of a bifurcated tubular prosthesis comprises creating a representation on a display of the contralateral stump opening and an electromagnetic marker coil, which is secured to an end portion of a guide member, and guiding the marker coil and guide member into the opening, while monitoring the relative positions of the contralateral stump opening representation and the marker representation on the display.

In another embodiment according to the invention, a method of cannulating the contralateral stump of a bifurcated tubular prosthesis comprises creating a representation on a display of the center of the contralateral stump opening and an electromagnetic marker coil that is secured to an endovascular delivery device, and guiding the marker and delivery device into the opening, while monitoring the relative positions of the opening center and the electromagnetic marker coil representations.

In another embodiment according to the invention, a method of cannulating the contralateral stump of a bifurcated tubular prosthesis comprises positioning one or more markers in the vicinity of the contralateral stump opening, displaying a representation on a display of the one or more markers and a tracking marker on one of a guidewire and a catheter, and guiding the tracking marker and one of the guidewire and catheter into the opening, while monitoring the relative position of the markers on the display.

Other features, advantages, and embodiments according to the invention will be apparent to those skilled in the art from the following description and accompanying drawings.

### BRIEF DESCRIPTION OF THE DRAWINGS

FIG. 1 diagrammatically illustrates one embodiment of a navigation system for cannulating the gate of a tubular prosthesis according to the invention.

FIGS. 2A-C diagrammatically illustrate cannulating an opening or gate of a tubular prosthesis, where FIG. 2A depicts a deployed bifurcated prosthesis and endovascular delivery of a guide device, having a target marker at its distal end, through one leg of the prosthesis and into to the opening or gate of the short leg of the prosthesis; FIG. 2B illustrates tracking a second guide device, having a marker at its distal end, toward the target marker at or in the vicinity of the short leg opening or gate; and FIG. 2C illustrates tracking a loaded stent-graft delivery catheter over the second guide device after the second guidewire has been cannulated and first guide member has been removed.

FIGS. 2D -E, diagrammatically illustrate one variation of the embodiment illustrated in FIGS. 2A-C, where FIG. 2D depicts guiding a diagnostic catheter having a marker at its distal end toward the target marker in the short leg opening or gate and into the short leg opening or gate and inserting a guidewire into the diagnostic catheter. FIG. 2E illustrates tracking a loaded stent-graft delivery catheter over the guidewire after the diagnostic catheter and first guide member with the target marker have been removed.

FIG. 2F illustrates the stent-graft delivered through the short leg opening or gate using either method shown in FIGS. 2A-C or 2D-E with the prosthesis fully deployed with stent-graft leg secured to the main body portion of the prosthesis.

FIG. 3A illustrates another marker arrangement for gate cannulation according to the invention.

FIG. 3B is and end view of the prosthesis shown in FIG. 3A taken along line 3B-3B.

FIG. 4 illustrates another marker embodiment to assist with cannulation.

FIG. 5 illustrates one display mode that can be used with the navigation system of FIG. 1 illustrating a display of a representation of an EMF coil superimposed on an image of the contralateral short leg of a bifurcated prosthesis in two different views.

FIG. 6 is a partial sectional view of a loaded stent-graft delivery catheter for delivering a stent-graft leg into the gate of the short leg of the main body portion of a bifurcated stent-graft.

FIG. 7 diagrammatically illustrates field generating and signal processing apparatus for locating electromagnetic markers.

FIG. 8A diagrammatically illustrates a known field generating and signal processing apparatus for locating leadless electromagnetic markers.

FIG. 8B is a diagrammatical section view of a known leadless electromagnetic marker.

### DETAILED DESCRIPTION

The following description will be made with reference to the drawings where when referring to the various figures, it should be understood that like numerals or characters indicate like elements.

Regarding proximal and distal positions, the proximal end of the prosthesis (e.g., stent-graft) is the end closest to the heart (by way of blood flow) whereas the distal end is the end farthest away from the heart during deployment. In contrast, the distal end of the catheter is usually identified as the end that is farthest from the operator, while the proximal end of the catheter is the end nearest the operator. Therefore, the prosthesis (e.g., stent-graft) and delivery system proximal and distal descriptions may be consistent or opposite to one another depending on prosthesis (e.g., stent-graft) location in relation to the catheter delivery path.

The invention generally involves, creating a target representation relating to an opening or gate in a tubular prosthesis area and tracking a device having a trackable element or marker toward the representation and into the opening, while monitoring the target representation and marker on a display.

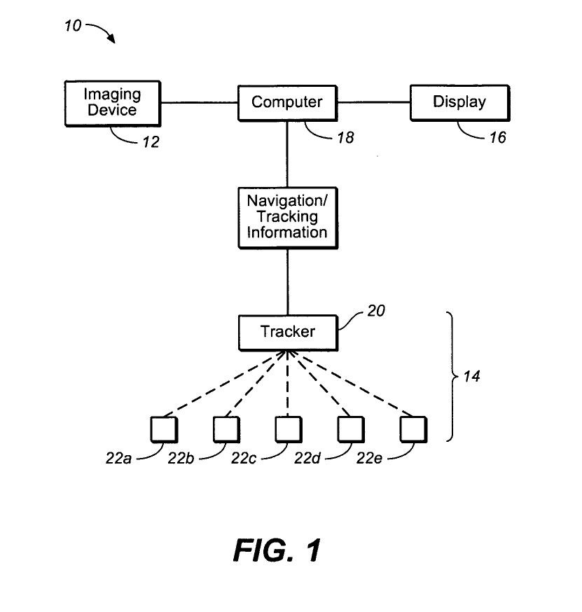

The method involves use of a navigation system and example of which is illustrated in FIG. 1 and generally designated with reference numeral 10. Navigation system 10 includes an imaging device 12) tracking system 14, which includes a tracker 20 and tracked elements 22a,b,c . . . n, which would, for example correspond to any of the markers described below, and a display coupled to computer or processor 18 to display information regarding the position and/or orientaton of one or more tracked elements, the device or devices to or with which one or more tracked elements are attached or associated, a representation of the an aspect of the prosthesis being cannulated, the relative positions of two or more of the tracked elements, or any combination thereof. In one example the tracking system tracks the position of the marker coils, processes the information received from the marker coils when they are activated, and provides five values for readimarker coil. Three values correspond to the XYZ coordinates in an XYZ coordinate system that correspond to the position of a marker coil in three-dimensional space. The fourth and fifth values for each marker indicate pitch and yaw of the marker coil, which are angular measurements of the marker coils in three-dimensional space that indicate the direction of the coils. The tracking system can be calibrated to track the center point of the marker coil as is known in the art. When the coil is a symmetrically configured cylinder as shown in the illustrative embodiments, all marker representations provided to the computer for processing and display are of the center point of the marker coil.

Imaging device 12, which can correspond to a pre-operative or intra-operative imaging device, is coupled to computer 18, which stores and processes the data that the imaging device acquires for display on display 16. Many known imaging systems can be used to acquire pre-operative or intra-operative data. One example of an imaging system that can be used to acquire pre-operative data is a CT scanner, which generates a three-dimensional (volumetric) image or model from a plurality of cross-sectional two-dimensional images. Another example of a scanner that can be used to acquire pre-operative data is a MR scanner, which also can provide a three-dimensional (volumetric) image. Regarding intra-operative data acquisition, navigation using a fluoroscopic two-dimensional system such as the virtual fluoroscopy system described in U.S. Pat. No. 6,470,207 entitled Navigational Guidance Via Computer-Assisted Fluoroscopic Imaging and which issued to Simon, et al., can be used. Alternatively, a fluoroscopic three-dimensional (volumetric) system such as the O-arm™ imaging system manufactured by Breakaway Imaging Inc. (Littleton, Mass.) can be used as well as other known imaging systems such as other fixed room fluoroscopes that are capable of three-dimensional reconstructions (e.g., Philips Allura with XperCT capability).

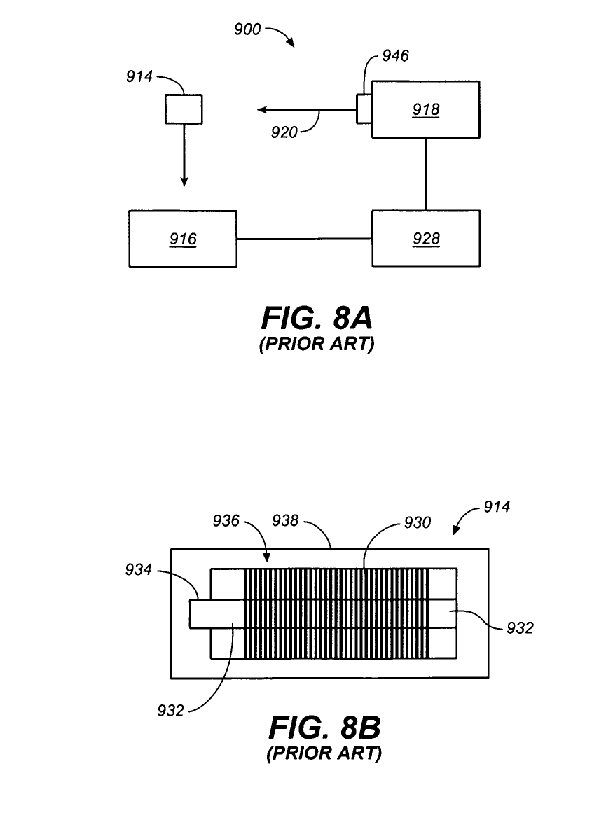

Tracking system 14, which measures positions and/or orientations, and which can, for example, incorporate known leadless tracking system 900, which is diagramatically shown in FIG. 8A and will be described in more detail below, provides navigational or tracking information to computer 18, which processes that information to display the representations of that information on display 16.

The tracking system typically comprises a tracker 20 and one or more tracked or trackable elements such as 22a, 22b, 22c, 22d, 22e . . . n, which correspond to any of the markers described below. The tracker provides navigation/tracking information to computer 18 so that the position and/or orientation of the marker coils in three-dimensional space can be displayed on display 16 with other marker coils or with a pre-acquired image or superimposed over a pre-acquired image.

When superimposing a tracking system data set over a pre-acquired data set, the data sets are registered. In one example, the pre-operative image can be registered via two-dimensional or three-dimensional fluoroscopy. For example, after the pre-operative data is acquired, a two-dimensional image is taken intra-operatively and is registered with the pre-operative image as is known in the art. Regarding registering two-dimensional and three dimensional images, see, for example, U.S. Patent Publication No. 2004/0215071 to Frank et al and entitled Method and Apparatus for Performing 2D to 3D Registration, the disclosure of which is hereby incorporated herein by reference in its entirety. In another example, an O-arm™ imaging system manufactured by Breakaway Imaging Inc. (Littleton, Mass.) can be used intra-operatively to take a picture/image of the navigation site to be navigated (see., e.g., U.S. Pat. No. 6,940,941, U.S. to Gregerson et al. and entitled Breakable Gantry Apparatus for Multidimensional X-Ray Based Imaging, U.S. Pat. No. 7,001,045 to Gregerson et al. and entitled Cantilevered Gantry Apparatus for X-Ray Imaging, U.S. Patent Publication No. 2004/0013225 to Gregerson et al. and entitled Systems and Methods for Imaging Large Field-of-View Objects, U.S. Patent Publication No. 2004/0013239 to Gregerson et al. and entitled Systems and Methods for Quasi-Simultaneous Multi-Planar X-Ray Imaging, U.S. Patent Publication No. 2004/0170254 to Gregerson et al. and entitled Gantry Positioning Apparatus for X-Ray Imaging, and U.S. Patent Publication No. 2004/0179643 to Gregerson et al. and entitled Apparatus and Method for Reconstruction of Volumetric Images in a Divergent Scanning Computed Tomography System, the disclosures of which are hereby incorporated by reference in their entirety). Another commercially available system for three-dimensional reconstruction of a volume space is the Innova® 3100 system built on GE's Revolution™ detector technology. A further representative system that performs image registration is described in U.S. Pat. No. 6,470,207 to Simon et al. and entitled Navigational Guidance Via Computer-Assisted Fluoroscopic Imaging, the disclosure of which is hereby incorporated herein by reference in its entirety.

When the markers are leadless resonating markers, they are inert, activatable devices that can be excited to generate a signal at a resonant frequency measurable by a sensor array that is remote from the marker as is known in the art. Such resonating markers generally comprise a core, coil windings, and a capacitor. The coil is wound around the core to form an inductor (L). The inductor (L) is connected to capacitor (C), so as to form a signal element. Accordingly, the signal element is an inductor (L) capacitor (C) resonant circuit. The coil wire typically is tightly wound around the core, which typically comprises ferromagnetic material, and can be formed from an elongated insulated copper wire (e.g., low resistance, small diameter, insulated wire). The resonating marker typically includes a protective encapsulation or casing to protect the signal element when tracked or implanted in a patient's body. The encapsulation or casing seals and/or encapsulates the signal element and can be made of plastic, glass, or other suitable inert material. The signal element can be potted with a silicone type plastic or covered with a thin heat shrink. A PTFE heat shrink also can be used to provide insulation and blood compatibility. The markers can have an axial dimension or length of approximately 2-14 mm and a diameter of approximately 0.5-5 mm. Further, the core can be provided with diametrically enlarged ferromagnetic end portions, which are not surrounded by coil wire, as described in U.S. Pat. No. 7,135,978 to Gisselberg et al. The end caps can have an outer diameter approximately the same as the outer diameter of the coil.

Methods for cannulating an opening or gate of a tubular prosthesis now will be described with reference to an imaging approach and an iconic representation approach. According to one navigation system embodiment using the imaging approach, representations of tracked elements and/or devices to which they are attached or associated are superimposed on pre-acquired anatomical images in real-time. “Pre-acquired,” as used herein, is not intended to imply any required minimum duration between receipt of the imaging information and displaying the corresponding image. Momentarily storing the corresponding imaging information (e.g., digital signals) in computer memory, while displaying the image (e.g., fluoroscopic image) constitutes pre-acquiring the image. The pre-acquired images can be acquired using fluoroscopic x-ray techniques, CT, MRI, or other known imaging modalities. Representations of markers and/or surgical or medical devices (e.g., catheters, probes, or prostheses) based on position information acquired from the tracking system can be overlaid on the pre-acquired images of the patient. In this manner, the physician is able to see the location of one or more markers relative to the deployed tubular prosthesis or an aspect of the deployed tubular prosthesis and/or the location of a surgical device to which one or more markers are attached relative to the deployed tubular prosthesis. A display of the prosthesis or an aspect of the prosthesis relative to the patient's anatomy in the vicinity of the prosthesis also can be displayed alone or in combination with any of the foregoing.

According to another navigation system embodiment, the navigation system provides, without the use of patient-specific medical images, the position of one or more tracked elements with iconic representations to indicate the positions and/or orientations of the tracked elements, the relative positions and/or orientations of the tracked elements when a plurality of tracked elements are used, the positions and/or orientations of the devices to which they are attached, or any combination thereof. And in other embodiments, such iconic representations can be displayed with or superimposed on patient-specific medical images. The iconic representations of the tracked elements do not correspond to images of the tracked elements, but rather graphics based on information corresponding to the position and/or orientation of the tracked elements.

### PROCEDURE EXAMPLE I

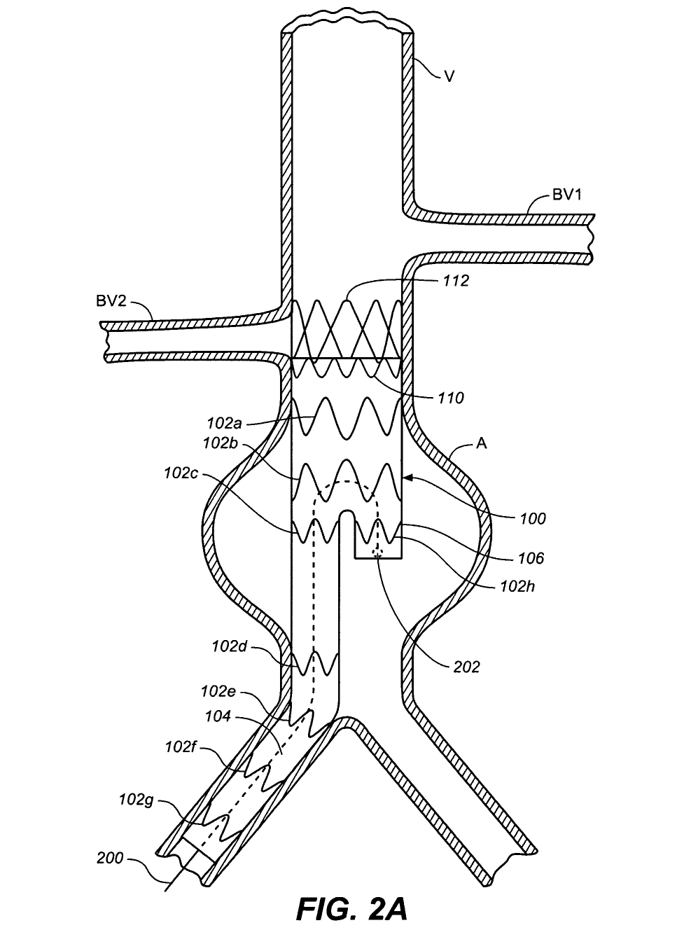

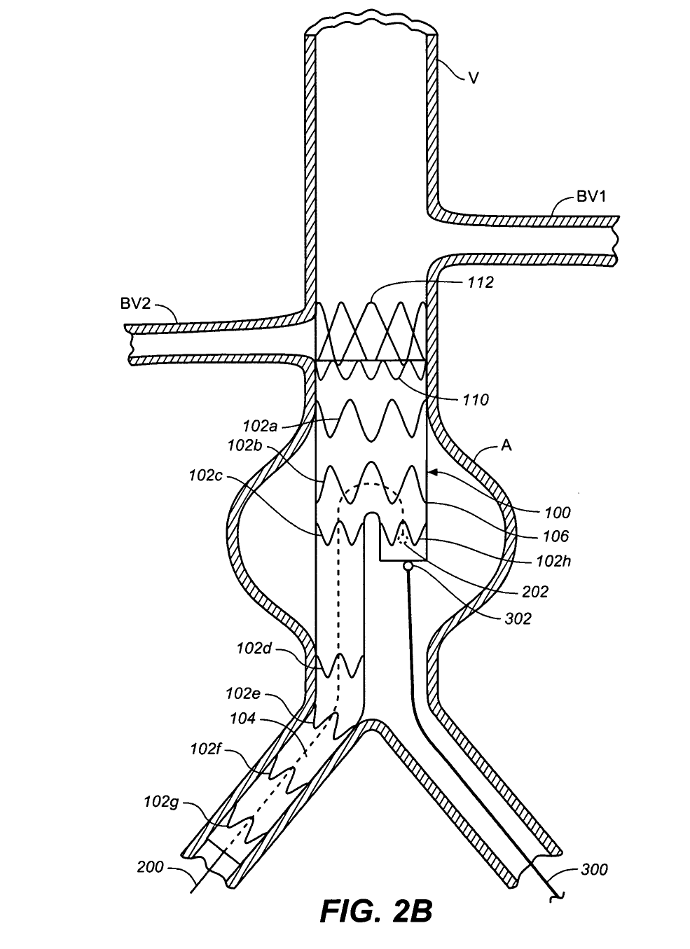

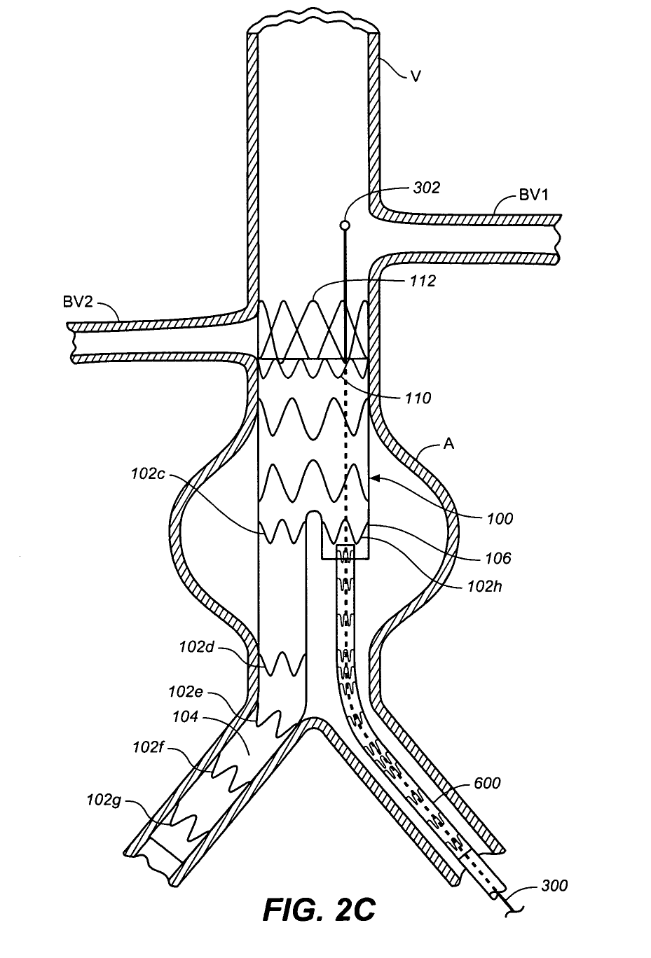

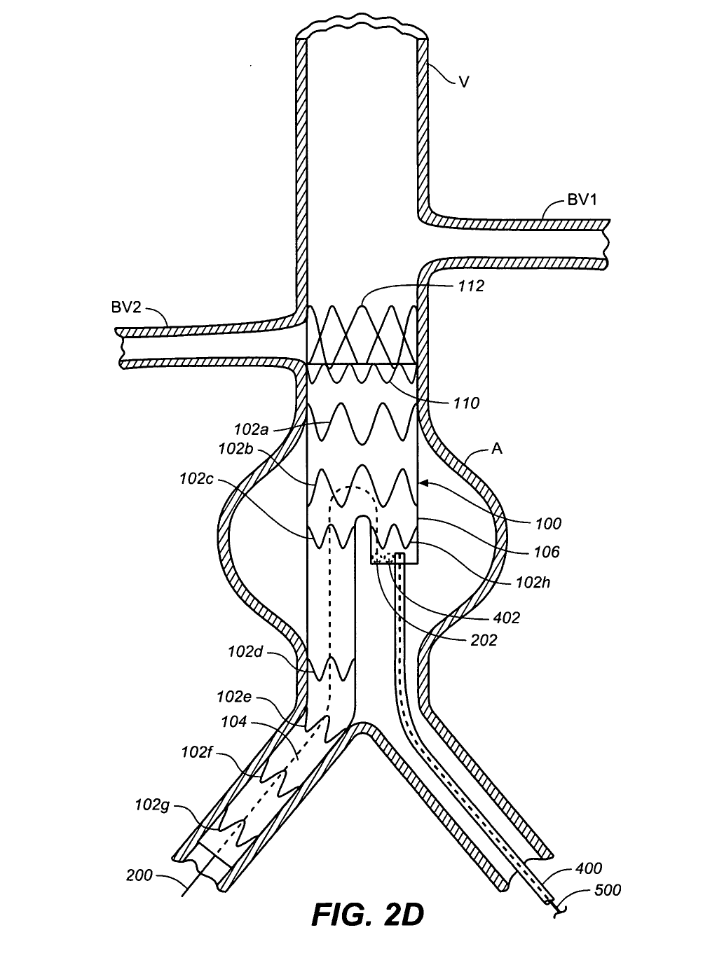

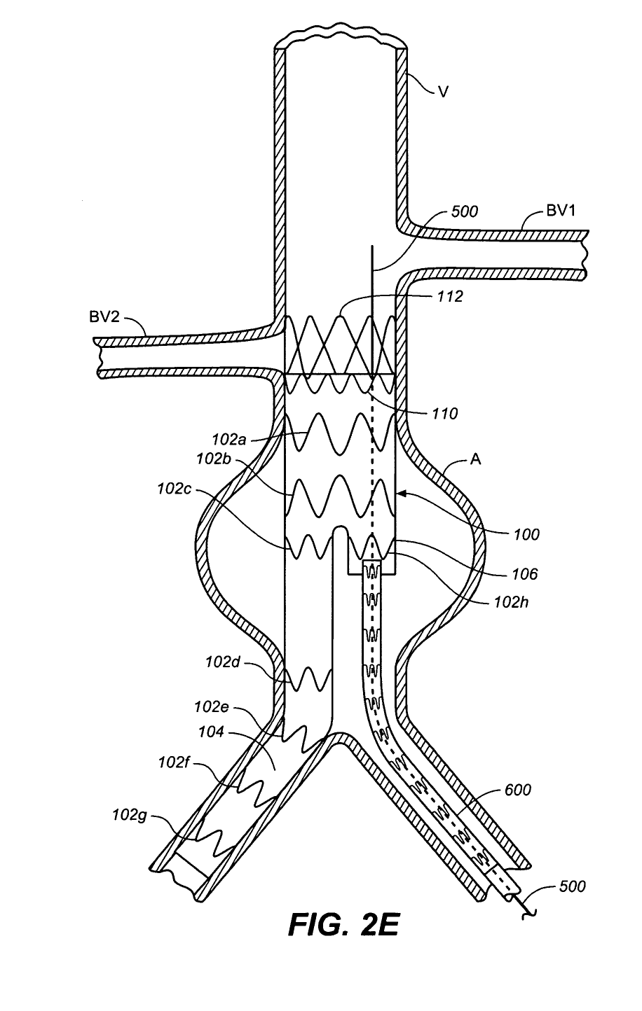

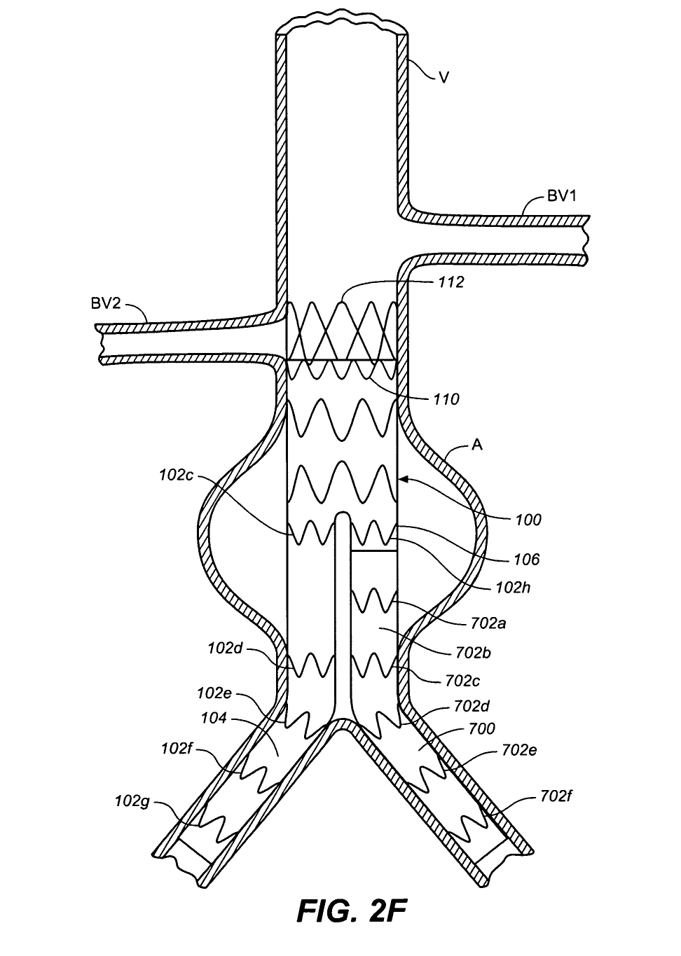

A first method of cannulating the gate will be described with reference to FIGS. 2A-C, which will be followed with a description of a variation, which will be described with reference to FIGS. 2D-E. In general, FIG. 2A depicts a deployed bifurcated prosthesis and endovascular delivery of a first elongated guide device (e.g., a guidewire, which has been delivered through a diagnostic catheter, or a steerable catheter), having a target marker at its distal end, through one leg of the prosthesis and into to the opening or gate of the short leg of the prosthesis. In another example, the first guide device can be a diagnostic or steerable catheter. FIG. 2B illustrates tracking a second elongated guide device, having a marker at its distal end, toward the target marker at or in the vicinity of the short leg opening or gate. FIG. 2C illustrates tracking a loaded stent-graft delivery catheter over the second guide device after the contralateral stump has been cannulated and the first guide device (e.g., a guidewire which has been delivered through a diagnostic or steerable catheter) has been removed. FIGS. 2D-E, diagrammatically illustrate one variation of the embodiment illustrated in FIGS. 2A-C, where FIG. 2D depicts guiding a steerable catheter having a marker at its distal end toward the target marker in the short leg opening or gate and into the short leg opening or gate and inserting a guidewire through the steerable catheter. FIG. 2E illustrates tracking a loaded stent-graft delivery catheter over the guidewire after the steerable catheter and first guide device with the target marker have been removed.

FIG. 2F illustrates the stent-graft delivered through the short leg opening or gate using either method shown in FIGS. 2A-C or 2D-E with the prosthesis fully deployed and with the contralateral stent-graft leg secured to the main body portion of the prosthesis. The procedure is described in more detail below.

A physician or interventionalist delivers the main body of a bifurcated stent-graft 100 using a traditional stent-graft delivery catheter to bypass aneurysm A in vessel V below branch vessels BV1 and BV2, which can correspond to the renal arteries, via the ipsilateral femoral artery as shown if FIG. 2A. The stent-graft main body section includes main body section 104, short leg section 106, and a plurality of undulating stent elements 102a-h, and an undulating radial support wire 110 all of which are covered with graft material. The stent-graft also can include traditional bare undulating wire 112 extending from the end adjacent branch vessel BV2. After contralateral short leg or stump 106 has been cannulated and the contralateral leg deployed as will be described below, the fully assembled stent-graft as shown in FIG. 2F includes the contralateral leg secured within contralateral short leg or stump 106 of stent-graft 100. The contralateral leg can include undulating stent elements 702a-f covered with graft material.

Returning to FIG. 2A, the physician or interventionalist introduces a guide device 200, which can be a guidewire delivered through a diagnostic catheter or steerable catheter. Tracked element 202, which can be a marker coil, is secured to the distal end of the guidewire, or along the side of the distal end of the diagnostic or steerable catheter in the case where a steerable catheter without a guidewire is used, through the ipsilateral femoral artery, into the iliac artery, and into the stent-graft. Tracked element or marker 202 can have the same construction as marker 914 or it can have the construction of another known or commercially available EMF (electromagnetic field) type coil marker. When the marker coil is on a guidewire, the physician or interventionalist will direct the guidewire distal end from the ipsilateral access point over the bifurcation and toward and into the contralateral stump to a position where the marker coil is at or substantially aligned with the stump opening. When the guide device is a guidewire delivered through a diagnostic catheter, the diagnostic catheter can have a steerable distal end to direct the guidewire toward the contralateral gate. Alternatively, the diagnostic catheter can have a pre-shaped end (e.g., Sheppard's hook shape) so that the diagnostic catheter can be arranged to point toward the contralateral gate and direct the guidewire toward the contralateral gate. In the arrangement where a guidewire is not used and, the marker coil is secured to the distal end of a steerable catheter, the physician or interventionalist similarly directs the catheter distal end over the bifurcation and toward and into the contralateral stump to position the marker coil at or to be substantially aligned with the stump opening or gate. Traditional fluoroscopic techniques can be used to place the marker coil at the desired location in the contralateral stump.

The tracking system is activated to generate electromagnetic energy in a volume of space in which the bifurcated stent-graft is positioned to excite coils positioned in that space so that the navigation system can provide the positional data of the coils in an XYZ coordinate system to the computer, which processes that information to display the position of the bifurcated stent-graft coil on the display.

Referring to FIG. 2B, a guidewire 300 (e.g., a steerable guidewire) with a marker coil 302 at its distal end is advanced toward marker coil 202 in the contralateral stump. Tracked element or marker 302 can have the same construction as marker 914 or it can have the construction of another known or commercially available EMF type coil marker. The tracking system (e.g., tracking system 14) provides data indicative of the positions and/or orientations of the coils in three-dimensional space in an XYZ coordinate system and that information is input into the computer (e.g., computer 18), which processes the information to display an iconic representation of the coils and their relative positions in three-dimensional space on a display such as display 16. The coil data is provided in real time so that the relative position of coils 202 and 302 can be monitored in real-time to assist the physician or interventionalist in cannulating the contralateral gate with the guidewire. Once the guidewire has cannulated the contralateral gate as shown in FIG. 2C, the physician or interventionalist tracks contralateral leg delivery catheter 600 over guidewire 300 and positions the distal end of the delivery catheter in the contralateral stump 106 of the main body section of the stent-graft using fluoroscopy.

Referring to FIGS. 2D-E an alternative procedure to above method for positioning a guidewire through the contralateral stump is shown. In this variation, the physician or interventionalist advances a steerable catheter 400, having a tracked element 402 secured to its distal or along the outer periphery of its distal end with glue or other suitable securing means, from the contralateral femoral artery toward marker coil 202 and into the contralateral gate where the target marker coil 202 is positioned. Tracked element or marker 402 can have the same construction as marker 914 or it can have the construction of another known or commercially available EMF type coil marker. The tracking system provides data indicative of the positions and/or orientations of the marker coils in three-dimensional space in an XYZ coordinate system and that information is input into the computer, which processes the information to display an iconic representation of the coils and their relative positions in three-dimensional space on display 16. The marker coil data is provided in real-time so that relative position of the coils can be monitored in real-time to assist the physician or interventionalist in cannulating the contralateral gate with the catheter. Once the catheter has cannulated contralateral stump 106, the physician or interventionalist advances guidewire 500 through the steerable catheter so that guidewire 500 is positioned in the stent-graft main body section as shown, for example, in FIG. 2E where the steerable catheter has been removed and contralateral stent-graft leg delivery catheter 600 tracked over guidewire 500, through the contralateral gate and into contralateral stump 106. The contralateral leg can be positioned using fluoroscopy.

As discussed above, when acquired navigational data indicative of the position and/or orientation of the marker coils is sent to computer or processor 18 for display, computer 18 can process that information to display a representation of the marker coils and their relative positions on display 16. Alternatively, the relative positions of the markers and the devices to which they are attached and the dimensions of those devices can be input into computer 18 so that computer 18 can process that information to display a representation of a respective device and its orientation.

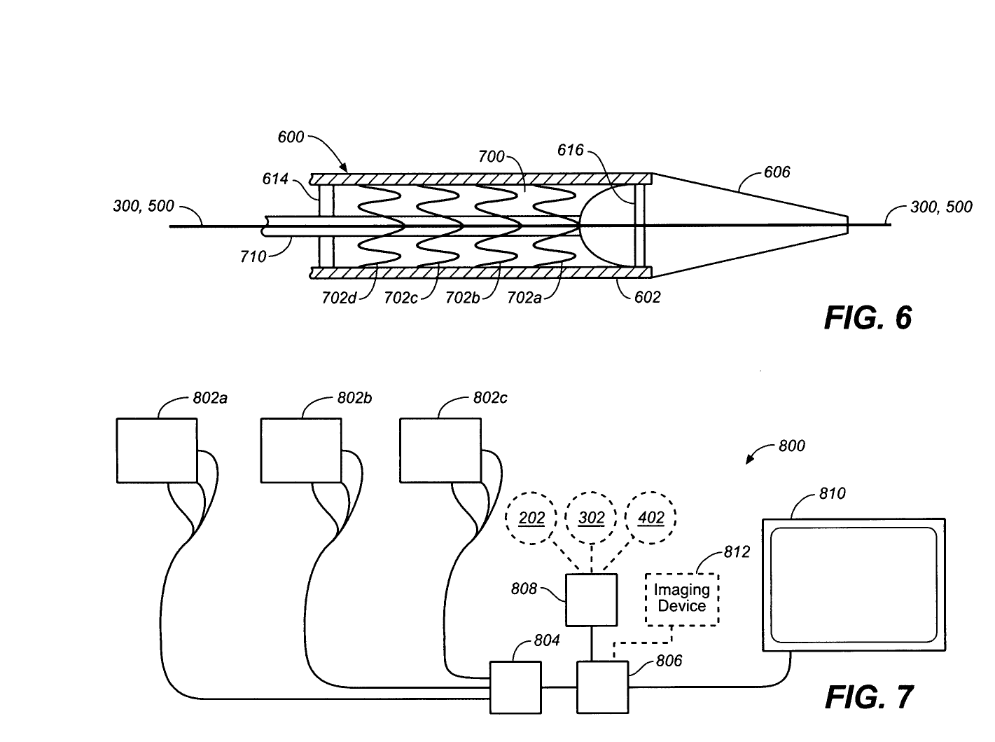

Referring to FIG. 6, one embodiment of delivery catheter 600 is shown. Delivery catheter 600 includes outer tuber 602 and a pusher disk 614 slidably disposed in outer tube 602 and surrounding and fixedly secured to guidewire tube 710. For purposes of illustration, the contralateral leg stent-graft 700 is shown loaded in delivery catheter 600, which can include tapered tip 606, which the guidewire tube can push outwardly away from the distal end of catheter tube 602 when deploying the stent-graft. Any other suitable delivery stent-graft delivery catheter system can be used such as the system described in U.S. Patent Application Publication No. 2004/0093063, which published on May 13, 2004 to Wright et al. and is entitled Controlled Deployment Delivery System,

Returning to the procedure, after the contralateral stent-graft leg is in the desired position in stump 106 using either approach described above, the contralateral leg is deployed and the delivery catheter and guidewire removed as depicted in FIG. 2F.

### PROCEDURE EXAMPLE II

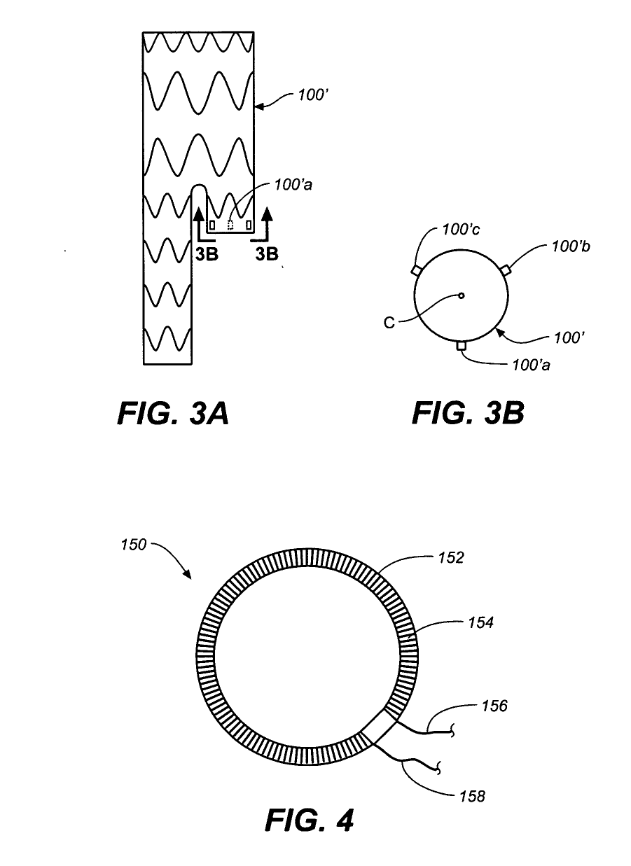

In this example, the bifurcated stent-graft main body that is delivered to bypass an aneurysm has a plurality of tracked elements secured to the outer periphery of its contralateral stump. In the embodiment shown in FIGS. 3A and B, one suitable bifurcated stent-graft main body section is shown and generally designated with reference numeral 100′. Bifurcated stent-graft 100′ is the same as the stent-graft main body section shown in FIG. 2A with the exception that it has tracked elements or marker coils 100′a, 100′b, and 100′c secured to the outer periphery of its contralateral stump and equidistantly spaced about the outer circumference of the contralateral stump. With this configuration, the positional data of these coils that is acquired by the tracking system and input into computer 18 can be processed to provide a target in the central region of the contralateral gate. The computer is input with the dimensions of the stent-graft, the positions and orientations of the marker coils relative to the stent-graft, and the coil dimensions. Accordingly, the computer can process acquired data regarding the direction (pitch and yaw) of the coils to determine the direction that the contralateral gate or opening faces e.g., relative to a reference such as vessel “V.” Tracked elements or marker coils 100′a, 100′b, and 100′c can have the same construction as marker 914 or they can have the construction of another known or commercially available EMF type coil marker. When they are EMF coils with leads coupled to a system that energizes the coils such as system 800 illustrated in FIG. 7, the leads can be cut using endoscopic tools after the contralateral gate has been cannulated. Alternatively, the coils can be provided with detachable leads as described in co-owned U.S. patent application Ser. No. 11/670,468, filed Feb. 2, 2007 and entitled Prosthesis Deployment Apparatus and Methods, the disclosure of which is hereby incorporated by reference herein in its entirety. Generally speaking, coils with leads can be made smaller in size than the leadless coils.

Returning to the procedure, the tracking system is set to generate electromagnetic energy in a volume of space in which the bifurcated stent-graft is positioned so that the navigation system can provide the positional data of the marker in an XYZ coordinate system to computer 18, which processes that information to display the position of the markers on the display.

In one method, the physician or interventionalist Introduces a guidewire, having an EMF coil fixed to its distal end as described above, into the contralateral femoral artery. The guidewire marker coil is advanced toward the target contralateral gate markers, while monitoring the relative position of the guidewire marker, target contralateral stump marker coils after the guidewire coil enters the excited volumetric space. The tracking system 14 provides data indicative of the positions and/or orientations of the markers in three dimensional space relative to an XYZ coordinate system and that information is input into the computer, which processes the information to display an iconic representation of the markers on the display. The tracking system provides real time data corresponding to the position of the markers as the guidewire is advanced so that the display can provide a real-time representation of the relative position of the markers in three-dimensional space to assist the physician in cannulating the contralateral gate with the guidewire.

After the physician positions the guidewire coil in the region surrounded by the contralateral stump coils and further advances it into the stent-graft main body section, a contralateral leg delivery catheter is tracked over the guidewire and positioned in the contralateral stump using fluoroscopy. The guidewire is removed and the contralateral leg deployed after which the delivery catheter is removed.

Alternatively, a steerable catheter, having a resonating marker secured to the outer surface of its distal end with glue or other suitable securing means, can be tracked toward the contralateral stump markers and advanced into the contralateral stump with the assistance of the display which displays the relative position of the marker coils. A guidewire and contralateral leg stent-graft delivery catheter can follow as described above in Example I. In a further alternative, the guidewire marker coil or catheter marker coil, whichever is used, can be displayed relative to a representation of the center of the contralateral gate and/or with a representation of the direction or orientation of the contralateral gate and the direction or orientation of the distal end of the guidewire or catheter since the dimensions of the distal end portions of the guidewire and/or catheter, the dimensions of their respective coils, and/or the relative positions or these devices as well as the orientation or direction of the coils relative to these devices can be input into the computer.

In yet a further variation, an annular coil such as coil 150 can replace coils 100′a, 100′b, and 100′c. Annular coil 150 is secured to the outer periphery of the contralateral stump with any suitable means such as sutures. This annular coil includes a tightly wound coil 154, which is encapsulated or encased within casing 152. Lead 156 extends from one coil end and lead 158 extends from the other coil end. Each lead extends through the encapsulation or casing and can be coupled to system such as system 800 as shown in FIG. 7. The leads can be detachable as described above. The coil ring configuration enables the tracking system to locate the position of the center of the ring and the direction of the ring.

### PROCEDURE EXAMPLE III

The main body portion of a bifurcated stent-graft is delivered to the target site for bypassing an aortic aneurysm as described above.

Before introducing the ipsilateral leg of the bifurcated stent-graft, an intra-operative three-dimensional image or data set of the bifurcated stent-graft contralateral gate (opening) and surrounding vasculature is acquired and input into the computer, which has navigation software to register the acquired data from the EMF coil tracker system with the coordinate system of the this intra-operative scan data set. As noted above, methods for registering the XYZ coordinates of the tracked elements in the coordinate system of the scanned bifurcated stent-graft contralateral stump are known in the art. In general, the XYZ coordinate system for the tracked elements and the XYZ coordinate system of the image of the scanned bifurcated stent-graft contralateral stump can be associated with an external reference location and through that association the coordinate systems of the tracked elements and the image of the scanned bifurcated stent-graft can be registered with one another using well known mathematical translations. One example is described in U.S. Patent Publication No. 2003073901, the disclosure of which is hereby incorporated herein by reference in its entirety. One commercially available example of a system that can provide such association and registration is the navigated O-arm™ Imaging System described above, which includes navigation software that registers similar coordinate systems. In this example, the image coordinate system of the imaging device, the O-arm™, is known (i.e., pre-calibrated) relative to an external reference location fixed relative to the imaging device (e.g., a set of markers or tracking devices such as EMF coils mounted on the imaging device). When the imaging device acquires a three-dimensional intra-operative image of the target zone, a position measurement is also acquired using the tracking system which measures the position and/or orientation (transformation, e.g., the determination of the three-dimensional position of an object relative to a patient is known in the art, and is discussed, for example, in the following references, each of which is hereby incorporated by reference: PCT Publication WO 96/11624 to Bucholz et al., published Apr. 25, 1996; U.S. Pat. No. 5,384,454 to Bucholz; U.S. Pat. No. 5,851,183 to Bucholz; and U.S. Pat. No. 5,871,445 to Bucholz. (a measurement of position and orientation of the device as defined by its markers, reference points, or tracking devices from an external coordinate system of the imaging device is a way of representing that postion and orientation) of the external coordinate system on the imaging device (e.g., defined by a set of markers or tracking devices such as EMF coils mounted to the imaging device) relative to the coordinate system (defined by the attached marker) on the main body portion of the stent. With the known location of the image coordinate system relative to the external reference location on the imaging device (from pre-calibration as noted above) and the location of the external reference location relative to the stent-graft, which was measured during image acquisition, straight-forward transformation mathematics results in the desired registration (i.e., the transformation between the physical stent-graft to the image coordinate system). And then the coordinate system of navigated marker is registered. One method for performing image registration is described in the previously mentioned publications to Bucholz.

Three-dimensional patient specific images can be registered to a patient on the operating room table (surgical space) using multiple two-dimensional image projections.

This process, which is often referred to as 2D/3D registration, uses two spatial transformations that can be established.

The first transformation is between the acquired fluoroscopic images and the three-dimensional image data set (e.g., CT or MR) corresponding to the same patient.

The second transformation is between the coordinate system of the fluoroscopic images and an externally measurable reference system attached to the fluoroscopic imager.

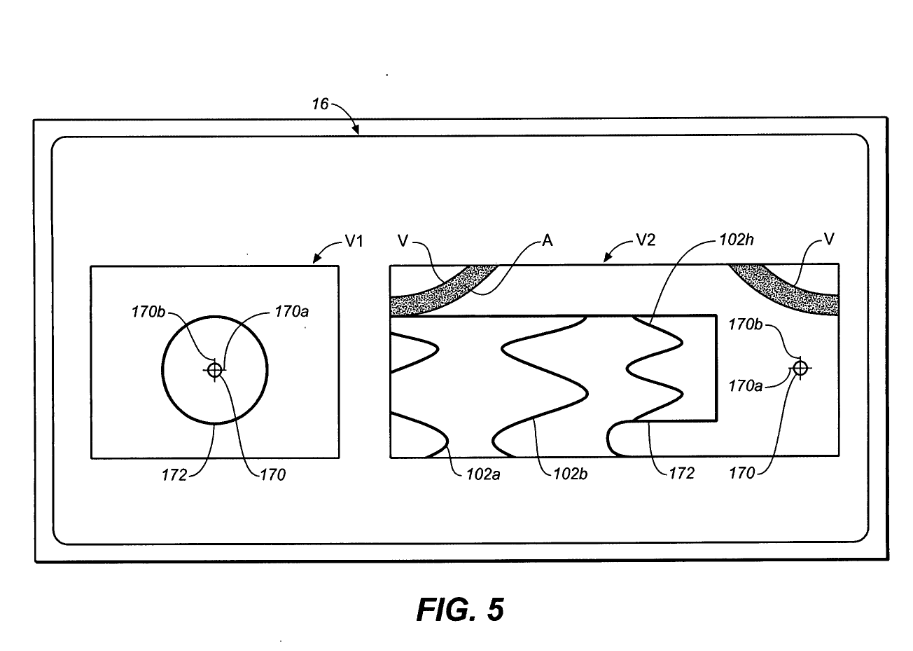

Once these transformations have been established, it is possible to directly relate surgical space to three-dimensional image space. A guidewire having an EMF marker coil at its distal end such as guidewire 200 or 300 is introduced through the femoral artery and advanced toward the contralateral gate. Alternatively, diagnostic catheter 400 having an EMF marker coil at its distal end is similarly advanced toward the contralateral gate. The tracking system is activated to generate electromagnetic energy in a volume of space in which the contralateral gate is positioned so that the tracking system can provide the positional data of the marker in an XYZ coordinate system to the computer when the marker coil enters that volumetric space. The data set acquired by the tracking system and corresponding to the position of the marker coil is input into the computer (e.g., computer 18) and registered with the data set acquired from the intra-operative scan of the contralateral gate to display a representation of the marker on the image of contralateral gate and surrounding region image as it approaches the contralateral gate to assist the physician in guiding the guidewire or diagnostic catheter into the contralateral gate. One example of such a display is shown in FIG. 5.

Based on tracked location of the marker coil on the guidewire or diagnostic or steerable catheter, it is possible to superimpose various graphical representations of the marker coil on the pre-acquired three dimensional image that was acquired intra-operatively. Referring to FIG. 5, one example of a display mode that can be used with the navigation system of FIG. 1 and which illustrates this method is shown depicting an iconic representation 170 of a tracked element, which in this example is an EMF coil, superimposed on an intra-operative image 172 of the contralateral short leg of a bifurcated prosthesis in two different views, V1 and V2. The iconic representation 170 of the tracked element does not correspond to an image of the tracked element, but rather graphics based on information corresponding to the position and/or orientation of the tracked element. View V1 is an end view of the contralateral short leg and view V2 is a lateral view of contralateral short leg. Specifically, view V1 shows a coil representation 170 of the tracked coil superimposed on the image of the contralateral short leg taken along the plane of the contralateral short leg opening, which also is referred to as the gate. View V2 shows an iconic representation 170 of the tracked coil laterally spaced from the contralateral gate of the contralateral short leg as the device to which the tracked coil is attached is guided toward the contralateral gate. In this example, the intersection of the cross-hairs 170a and 170b are indicative of the position of the tracked coil relative to the contralateral gate. The iconic representations can be provided continuously in real time and superimposed on the contralateral short leg image to assist the physician or interventionalist in cannulating the contralateral gate with the device to which the tracked coil is attached.

Due to the relatively small size of the guidewire or catheter, either can still cannulate the contralateral gate when guided to the contralateral gate based on the intra-operative image of the contralateral gate even when the contralateral leg moves from where it was when the image was taken due to normal physiological activity. If the contralateral gate moves laterally about 3-4 mm, the guidewire or catheter would still cannulate the contralateral gate. However, the image of the contralateral gate can be updated if desired. Alternatively, a marker coil can be added to the contralateral gate to allow tracking of this movement. A two dimensional fluoroscopic update typically may require one or two seconds and a three-dimensional fluoroscopic update typically may require 30-60 seconds depending on resolution.

The display illustrated in FIG. 5 is one example of displaying an iconic representation of a marker coil superimposed on an image generated from a pre-acquired data set that was obtained from a scan. The example illustrates indexing real time data acquired from the guidewire marker against the non-real time anatomical image so that the physician or interventionalist may understand a device location relative to the previously acquired anatomical image. In the embodiment illustrated in FIG. 5, the images correspond to two two-dimensional stent-graft images in-vivo. However, other displays can be used.

In one embodiment, a three-dimensional perspective view representative of the intra-operative scan can be displayed and the marker coil superimposed on the three-dimensional view. In one variation of this embodiment, the guidewire to which the marker coil is attached is superimposed on the pre-acquired three-dimensional perspective view.

In another embodiment, the display can show a CAD representation of the tracked marker superimposed on the pre-acquired, registered, three-dimensional image. The CAD representation is another form an iconic representation. In one variation, the CAD representation can be overlaid on a previously acquired, “registered,” two-dimensional image. The virtual location of the tracked marker that the CAD representation depicts is interposed within or superimposed on the pre-acquired data set and thus permit the physician or interventionalist to more simply understand where within the patient's anatomy the device to which the marker is attached is located. This also assists with gate cannulation.

Regarding activating magnetically sensitive, electrically conductive marker coils, prespecified electromagnetic fields are projected to the portion of the anatomical structure of interest (e.g., that portion that includes all prospective locations of the coils and/or device(s)) in a manner and sufficient to induce voltage signals in the coil(s). Electrical measurements of the voltage signals are sufficient to compute the angular orientation and positional coordinates of the sensing coil(s) and hence the location and/or orientation of coils and or objects to which they are associated. An example of sensing coils for determining the location of a catheter or endoscopic probe inserted into a selected body cavity of a patient undergoing surgery in response to prespecified electromagnetic fields is disclosed in U.S. Pat. No. 5,592,939 to Martinelli, the disclosure of which is hereby incorporated herein by reference in its entirety. Another example of methods and apparatus for locating the position in three dimensions of a sensor coil by generating magnetic fields which are detected at the sensor is disclosed in U.S. Pat. No. 5,913,820 to Bladen, et al., the disclosure of which is hereby incorporated herein by reference in its entirety.

Referring to FIG. 7, one navigation system including a field generating and signal processing circuit configuration for generating magnetic fields at the location of marker coils when the marker coils are magnetically sensitive, electrically conductive coils having leads, and processing the voltage signals that the markers generate in response to the generated magnetic fields is shown and generally designated with reference numeral 800. Although nine coils are shown in three groups of three in the example depicted in FIG. 7, it should be understood that nine separate coils can be used. More generally, the product (multiplication) of the number of receiver coils and the number of transmitter coils must equal at least nine. So for example, it is possible to have three transmitter coils and three receiver coils to measure six degrees of freedom.

In the illustrated example, circuit 800 generally includes three electromagnetic field (EMF) generators 802a, 802b, and 802c, amplifier 804, controller 806, measurement unit 808, and display device 810. Each field generator comprises three electrically separate coils of wire (generating coils) wound about a cuboid wooden former. The three coils of each field generator are wound so that the axes of the coils are mutually perpendicular. The nine generating coils are separately electrically connected to amplifier 804, which is able, under the direction of controller 806, to drive each coil individually.

In use, controller 806 directs amplifier 804 to drive each of the nine generating coils sequentially. Once the quasi-static field from a particular generating coil is established, the value of the voltage induced in each coil by this field is measured by the measurement unit 808, processed and passed to controller 806, which stores the value and then instructs the amplifier 804 to stop driving the present generating coil and to start driving the next generating coil. When all generating coils have been driven, or energized, and the corresponding nine voltages induced into each sensing coil have been measured and stored, controller 806, which can correspond to computer 18 calculates the location and orientation of each sensor relative to the field generators and displays this on a display device 810, which can correspond to display device 16. This calculation can be carried out while the subsequent set of nine measurements are being taken. Thus, by sequentially driving each of the nine generating coils, arranged in three groups of three mutually orthogonal coils, the location and orientation of each sensing coil can be determined.

The sensor and generating coil specifications, as well as the processing steps are within the skill of one of ordinary skill of the art. An example of coil specifications and general processing steps that can be used are disclosed in U.S. Pat. No. 5,913,820 to Bladen, et al. (supra).

FIGS. 7A and 7B illustrate a system and components for generating an excitation signal for activating a leadless resonating marker assembly and locating the marker in three-dimensional space which can be used in systems for performing methods in accordance with aspects of the present invention.

Referring to FIG. 8A, a known leadless electromagnetic system is shown. FIG. 8A is a schematic view of a system 900 for energizing and locating one or more leadless resonating marker assemblies 914 in three-dimensional space relative to a sensor array 916 where one marker assembly 914 is shown in this example. System 900 includes a source generator 918 that generates a selected magnetic excitation field or excitation signal 920 that energizes each marker assembly 914. Each energized marker assembly 914 generates a measurable marker signal 922 that can be sufficiently measured in the presence of both the excitation source signal and environmental noise sources. The marker assemblies 914 can be positioned in or on a selected object in a known orientation relative to each other. The marker signals 922 are measured by a plurality of sensors (not shown) sensor array 916. The sensors 926 are coupled to a signal processor 928 that utilizes the measurement of the marker signals 922 from the sensors 926 to calculate the location of each marker assembly 914 in three-dimensional space relative to a known frame of reference, such as the sensor array 916.

Source generator 918 is configured to generate the excitation signal 920 so that one or more marker assemblies 914 are sufficiently energized to generate the marker signals 922. The source generator 918 can be switched off after the marker assemblies are energized. Once the source generator 918 is switched off, the excitation signal 920 terminates and is not measurable. Accordingly, sensors 926 in sensor array 916 will receive only marker signals 922 without any interference or magnetic field distortion induced by the excitation signal 920. Termination of the excitation signal 920 occurs before a measurement phase in which marker signals 922 are measured. Such termination of the excitation signal before the measurement phase when the energized marker assemblies 914 are generating the marker signals 922 allows for a sensor array 916 of increased sensitivity that can provide data of a high signal-to-noise ratio to the signal processor 928 for extremely accurate determination of the three-dimensional location of the marker assemblies 914 relative to the sensor array or other frame of reference.

The miniature marker assemblies 914 in the system 900 are inert, activatable assemblies that can be excited to generate a signal at a resonant frequency measurable by the sensor array 916 remote from the target on which they are placed. The miniature marker assemblies 914 have, as one example, a diameter of approximately 2 mm and a length of approximately 5 mm, although other marker assemblies can have different dimensions as described above. An example of such a marker detection systems are described in detail in U.S. Patent Publication No. 20020193685 entitled Guided Radiation Therapy System, filed Jun. 8, 2001 and published on Dec. 19, 2002, and U.S. Pat. No. 6,822,570 to Dimmer et al., entitled System For Spacially Adjustable Excitation Of Leadless Miniature Marker, all of the disclosures of which are incorporated herein in their entirety by reference thereto.

Referring to FIG. 9B, the illustrated marker assembly 914 includes a coil 930 wound around a ferromagnetic core 932 to form an inductor (L). The inductor (L) is connected to a capacitor 934, so as to form a signal element 936. Accordingly, the signal element 836 is an inductor (L) capacitor (C) resonant circuit. The signal element 936 can be enclosed and sealed in an encapsulation member 938 made of plastic, glass, or other inert material. The illustrated marker assembly 914 is a fully contained and inert unit that can be used, as an example, in medical procedures in which the marker assembly is secured on and/or implanted in a patient's body as described in U.S. Pat. No. 6,822,570 (supra).

The marker assembly 914 is energized, and thus activated, by the magnetic excitation field or excitation signal 920 generated by the source generator 918 such that the marker's signal element 936 generates the measurable marker signal 922. The strength of the measurable marker signal 922 is high relative to environmental background noise at the marker resonant frequency, thereby allowing the marker assembly 914 to be precisely located in three-dimensional space relative to sensor array 916.

The source generator 918 can be adjustable to generate a magnetic field 920 having a waveform that contains energy at selected frequencies that substantially match the resonant frequency of the specifically tuned marker assembly 914. When the marker assembly 914 is excited by the magnetic field 920, the signal element 936 generates the response marker signal 922 containing frequency components centered at the marker's resonant frequency. After the marker assembly 914 us energized for a selected time period, the source generator 918 is switched to the “off” position so the pulsed excitation signal 920 is terminated and provided no measurable interference with the marker signal 922 as received by the sensor array 916.

The marker assembly 914 is constructed to provide an appropriately strong and distinct signal by optimizing marker characteristics and by accurately tuning the marker assembly to a predetermined frequency. Accordingly, multiple uniquely tuned, energized marker assemblies 914 may be reliably and uniquely measured by the sensor array 916. The unique marker assemblies 914 at unique resonant frequencies may be excited and measured simultaneously or during unique time periods. The signal from the tuned miniature marker assembly 914 is significantly above environmental signal noise and sufficiently strong to allow the signal processor 928 (FIG. 7A) to determine the marker assembly's identity, precise location, and orientation in three dimensional space relative to the sensor array 916 or other selected reference frame.

A system corresponding to system 900 is described in U.S. Pat. No. 6,822,570 to Dimmer et al., entitled System For Spacially Adjustable Excitation Of Leadless Miniature Marker and which was filed Aug. 7, 2002, the entire disclosure of which is hereby incorporated herein in its entirety by reference thereto. According to U.S. Pat. No. 6,822,570, the system can be used in many different applications in which the miniature marker's precise three-dimensional location within an accuracy of approximately 1 mm can be uniquely identified within a relatively large navigational or excitation volume, such as a volume of 12 cm×12 cm×12 cm or greater. One such application is the use of the system to accurately track the position of targets (e.g., tissue) within the human body. In this application, the leadless marker assemblies are implanted at or near the target so the marker assemblies move with the target as a unit and provide positional references of the target relative to a reference frame outside of the body. U.S. Pat. No. 6,822,570 further notes that such a system could also track relative positions of therapeutic devices (i.e., surgical tools, tissue, ablation devices, radiation delivery devices, or other medical devices) relative to the same fixed reference frame by positioning additional leadless marker assemblies on these devices at known locations or by positioning these devices relative to the reference frame. The size of the leadless markers used on therapeutic devices may be increased to allow for greater marker signal levels and a corresponding increase in navigational volume for these devices.

Other examples of leadless markers and/or devices for generating magnetic excitation fields and sensing the target signal are disclosed in U.S. Patent Publication No. 20030052785 to Gisselberg et al. and entitled Miniature Resonating Marker Assembly, U.S. Pat. No. 7,135,978 to Gisselberg et al. and entitled Miniature Resonating Marker Assembly, U.S. Pat. No. 6,889,833 to Seiler et al. and entitled Packaged Systems For Implanting Markers In A Patient And Methods For Manufacturing And Using Such Systems, U.S. Pat. No. 6,812,842 to Dimmer and entitled Systems For Excitation Of Leadless Miniature Marker, U.S. Pat. No. 6,838,990 to Dimmer and entitled Systems For Excitation Of Leadless Miniature Marker, U.S. Pat. No. 6,977,504 to Wright et al. and entitled Receiver Used In Marker Localization Sensing System Using Coherent Detection, U.S. Pat. No. 7,026,927 to Wright et al. and entitled Receiver Used In Marker Localization Sensing System And Having Dithering In Excitation all the disclosures of which are hereby incorporated herein in their entirety by reference thereto.

Other example of a suitable leadless marker construction and system is the Calypso® 4D Localization System, which is a target localization platform based on detection of AC electromagnetic markers, called Beacon® transponders, which are implantable devices. These localization systems and markers have been developed by Calypso® Medical Technologies (Seattle, Wash.).

Any feature described in any one embodiment described herein can be combined with any other feature of any of the other embodiments whether preferred or not.

Variations and modifications of the devices and methods disclosed herein will be readily apparent to persons skilled in the art.

What is claimed is:

1. A method of cannulating the contralateral stump of a bifurcated tubular prosthesis comprising creating a representation on a display of the contralateral stump opening and an electromagnetic marker coil, which is secured to an end portion of a guide member, and guiding the marker coil and guide member into the opening, while monitoring the relative positions of the contralateral stump opening representation and the marker representation on the display.

2. The method of claim 1 wherein the guide member is guidewire.

3. The method of claim 1 wherein the guide member is a catheter.

4. The method of claim 1 wherein the guide member is a catheter.

5. A method of cannulating the contralateral stump of a bifurcated tubular prosthesis comprising creating a representation on a display of the center of the contralateral stump opening and an electromagnetic marker coil that is secured to an endovascular delivery device, and guiding the marker and delivery device into the opening, while monitoring the relative positions of the opening center and the electromagnetic marker coil representations.

6. The method of claim 5 wherein the center and direction of the contralateral stump is determined using electromagnetic field coils.

7. A method of cannulating the contralateral stump of a bifurcated tubular prosthesis comprising positioning one or more markers in the vicinity of the contralateral stump opening, displaying a representation on a display of the one or more markers and a tracking marker on one of a guidewire and a catheter, and guiding the tracking marker and one of the guidewire and catheter into the opening, while monitoring the relative position of the markers on the display.
